(19) United States
(12) Patent Application Publication
(10) Pub. No.: US 2023/0204348 A1
(43) Pub. Date: Jun. 29, 2023

(54) THREE DIMENSIONAL SCANNING APPARATUS

(71) Applicant: QISDA CORPORATION, Taoyuan City (TW)

(72) Inventors: Ching-Huey Wang, Taoyuan City (TW); Tsung-Hsun Wu, Taoyuan City (TW); Chih-Ming Hu, Taoyuan City (TW)

(73) Assignee: QISDA CORPORATION, Taoyuan City (TW)

(21) Appl. No.: 17/736,079

(22) Filed: May 3, 2022

(30) Foreign Application Priority Data
Dec. 28, 2021 (CN) 202111622352.7

(51) Int. Cl. G01B 11/25

(52) U.S. Cl. CPC G01B 11/2518; A61C 9/006

(57) ABSTRACT

A three dimensional scanning apparatus is used to detect a contour of an object, and includes an illumination light source, a first elliptic opening portion, a reference pattern generator, a second elliptic opening portion and an optical receiver. The illumination light source emits an illumination beam. The reference pattern generator provides a reference pattern by projection of the illumination beam, and transmits the reference pattern toward the object via the first elliptic opening portion. The optical receiver receives a detection pattern reflected from the object via the second elliptic opening portion, so as to analyze a difference between the reference pattern and the detection pattern for acquiring the contour.

### BACKGROUND OF THE INVENTION

### 1. Field of the Invention

The present invention relates to a three dimensional scanning apparatus, and more particularly, to a three dimensional scanning apparatus of keeping the scanning depth of field and the pattern's intensity.

### 2. Description of the Prior Art

With the advanced technology, optical scanning technology is widely used in various fields, such as the oral scanner. The conventional optical oral scanner utilizes a light source to project illumination light onto the grating, and the grating pattern reflected by the grating is projected onto the target object through the first aperture. The target object then reflects the grating pattern to pass through the second aperture and be received by the image capturing device. However, one of the first aperture and the second aperture of the conventional optical oral scanner is a non-elliptical pinhole, which results in low image capturing quality due to effect of the amount of light received by the image capturing device. Therefore, design of an optical scanning system capable of keeping the scanning depth of field and image intensity in the low illumination environment is an important issue in the related optical design industry.

### SUMMARY OF THE INVENTION

The present invention provides a three dimensional scanning apparatus of keeping the scanning depth of field and the pattern's intensity for solving above drawbacks.

According to the claimed invention, a three dimensional scanning apparatus is used to detect a contour of an object. The three dimensional scanning apparatus includes an illumination light source, a first elliptic opening portion, a reference pattern generator, a second elliptic opening portion and an optical receiver. The illumination light source is adapted to emit an illumination beam. The reference pattern generator is adapted to provide a reference pattern via the illumination beam and transmit the reference pattern toward the object through the first elliptic opening portion. The optical receiver is adapted to receive a detection pattern reflected from the object via the second elliptic opening portion, so as to analyze a difference between the reference pattern and the detection pattern for acquiring the contour.

According to the claimed invention, a difference between a first ratio of a major axis to a minor axis of the first elliptic opening portion and a second ratio of a major axis to a minor axis of the second elliptic opening portion is smaller than a predefined threshold. Or, a first included angle of one of a major axis and a minor axis of the first elliptic opening portion relative to the reference pattern is the same as or similar to a second included angle of one of a major axis and a minor axis of the second elliptic opening portion relative to the reference pattern.

According to the claimed invention, the reference pattern comprises a plurality of stripes arranged adjacent to each other, and an axis direction of a major axis of the first elliptic opening portion is crossed by an arrangement direction of the plurality of stripes. Besides, the reference pattern comprises a plurality of stripes arranged adjacent to each other, and an axis direction of a major axis of the second elliptic opening portion is crossed by an arrangement direction of the plurality of stripes. An included angle between the major axis of the first elliptic opening portion or the second elliptic opening portion and the arrangement direction is equal to ninety degrees, or the major axis of the first elliptic opening portion or the second elliptic opening portion is perpendicular to the arrangement direction and has an angle error tolerance.

According to the claimed invention, the first elliptic opening portion and the second elliptic opening portion are fixed aperture opening portions or variable aperture opening portions. The reference pattern generator is an assembly of digital mirror devices, and the reference pattern is a predefined granting pattern generated by the assembly of digital mirror devices.

According to the claimed invention, the reference pattern comprises a plurality of stripes arranged adjacent to each other, at least one of a major axis of the first elliptic opening portion and a major axis of the second elliptic opening portion is lengthened along a stripe direction of the plurality of stripes, so as to keep a scanning depth of field of the three dimensional scanning apparatus and increase an intensity of the detection pattern.

According to the claimed invention, the reference pattern comprises a plurality of stripes arranged adjacent to each other, at least one of a minor axis of the first elliptic opening portion and a minor axis of the second elliptic opening portion is shortened along an arrangement direction of the plurality of stripes, so as to keep an intensity of the detection pattern and increase a scanning depth of field of the three dimensional scanning apparatus.

The three dimensional scanning apparatus of the present invention can be used in the optical molding apparatus in the dental department. A light source cannot put into the mouth of the patient, and an illumination efficiency of an external light source for projecting into the patient's mouth is insufficient, so that the optical system of the optical molding apparatus has to be advanced for preferred detection accuracy. The three dimensional scanning apparatus of the present invention can adjust the shape, the ratio and/or the size of the opening portion on the aperture unit to keep the scanning depth of field and the intensity of the pattern, so as to acquire the accurate and clear contour of the object.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram of a three dimensional scanning apparatus according to an embodiment of the present invention.

FIG. 2 is a diagram of the first elliptic opening portion and the second elliptic opening portion according to the embodiment of the present invention.

FIG. 3 is a diagram of the reference pattern, the first elliptic opening portion and the second elliptic opening portion according to the embodiment of the present invention.

FIG. 4 is a diagram of the first elliptic opening portion according to another embodiment of the present invention.

### DETAILED DESCRIPTION

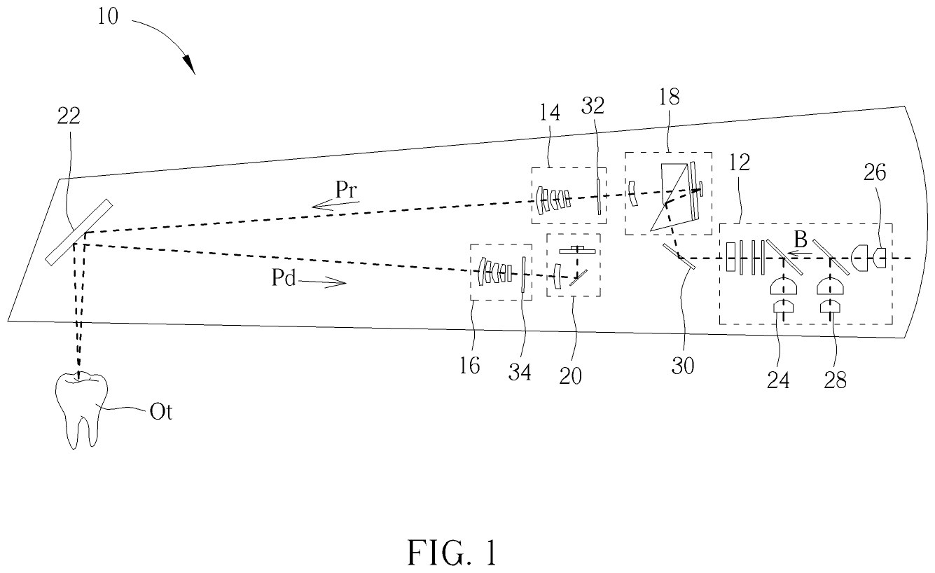

Please refer to FIG. 1. FIG. 1 is a diagram of a three dimensional scanning apparatus 10 according to an embodiment of the present invention. The three dimensional scanning apparatus 10 can utilize optical detection technology to detect a contour of an object Ot. In the embodiment of the present invention, the object Ot can be a teeth, and the three dimensional scanning apparatus 10 can be an optical molding apparatus in the dental department, which depend on an actual demand. The three dimensional scanning apparatus 10 can include an illumination light source 12, a first optical module 14, a second optical module 16, a reference pattern generator 18 and an optical receiver 20. The illumination light source 12 can emit an illumination beam B to pass through the reference pattern generator 18 for generating a reference pattern Pr. The reference pattern Pr can be projected onto the object Ot via the first optical module 14 and a reflector 22. A detection pattern Pd reflected by the object Ot can pass through the second optical module 16 to be received by the optical receiver 20 and then transformed into the contour of the object Ot.

The illumination light source 12 can include three lighting units 24, 26 and 28, which respectively are a red light emitting diode, a blue light emitting diode and a green light emitting diode; application of the lighting units 24, 26 and 28 depends on a design demand, and other possible variation is omitted herein for simplicity. The illumination light source 12 may further include other optical components, such as an optical reflecting component, an optical filtering component, an optical diffusing component and an optical splitting component. A number, a property, a position and an arrangement angle of the optical components are defined in accordance with illumination features of the lighting units 24, 26 and 28. The illumination beam B emitted from the illumination light source 12 can be transmitted to the reference pattern generator 18 optionally via a reflector 30.

In the embodiment of the present invention, the reference pattern generator 18 can be an assembly of digital mirror devices, which includes a plurality of micro mirrors arranged as an array. The plurality of micro mirrors can generate a predefined granting pattern for being the reference pattern Pr in accordance with a control command. For example, the assembly of digital mirror devices can control the plurality of micro mirrors in odd rows or columns, or the plurality of micro mirrors in even rows or columns, or the plurality of micro mirrors in specific position to provide reflection function, and other unmentioned micro mirrors are rotated and cannot reflect the illumination beam B, so as to generate the predefined granting pattern. The reference pattern generator 18 may be other optical component or any possible non-optical component, which depends on the design demand.

The first optical module 14 and the second optical module 16 can respectively include a plurality of optical components and a plurality of aperture units. The optical components can be any kinds of convex lenses and concave lenses. The aperture units can be structural components with an elliptical opening portion. The first optical module 14 can at least include a first elliptic opening portion 32, and the second optical module 16 can at least include a second elliptic opening portion 34. The first elliptic opening portion 32 and the second elliptic opening portion 34 can be respectively disposed on the aperture unit of the related optical module. The illumination beam B on the non-focal plane can be eliminated by the first elliptic opening portion 32 and the second elliptic opening portion 34, and the clear reference pattern Pr and the clear detection pattern Pd can be generated accordingly. The optical receiver 20 can be a common image capturing component, such as the charge coupled device (CCD) or the complementary metal oxide semiconductor (CMOS), which depends on the actual demand.

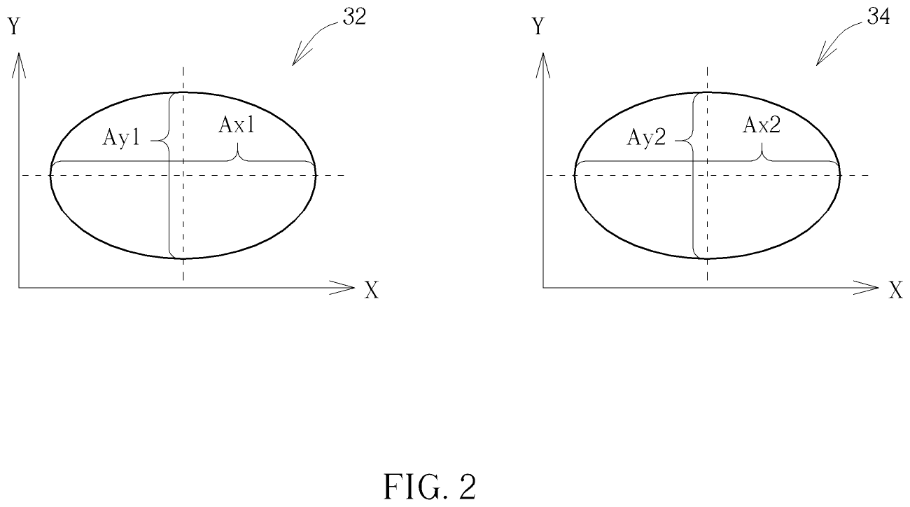

Please refer to FIG. 2. FIG. 2 is a diagram of the first elliptic opening portion 32 and the second elliptic opening portion 34 according to the embodiment of the present invention. The first elliptic opening portion 32 can have a first major axis Ax1 and a first minor axis Ay1. The second elliptic opening portion 34 can have a second major axis Ax2 and a second minor axis Ay2. A difference between a first ratio of the first major axis Ax1 to the first minor axis Ay1 and a second ratio of the second major axis Ax2 to the second minor axis Ay2 can be smaller than a predefined threshold. A possible range of the predefined threshold may be set between 1˜10%, which depends on the actual demand. The present invention can preset the difference between the first ratio and the second ratio, so as to define that the first elliptic opening portion 32 and the second elliptic opening portion 34 belong to two oval holes having the same or similar shapes and the same or similar ratio of the major axis to the minor axis.

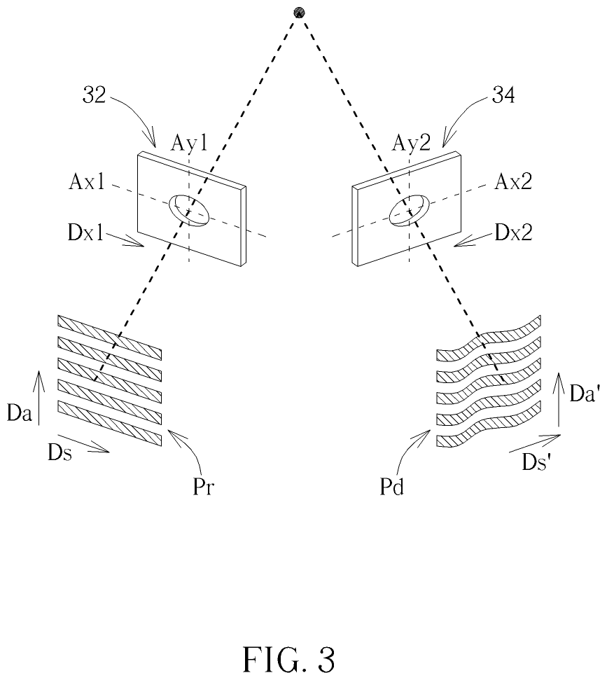

Please refer to FIG. 3. FIG. 3 is a diagram of the reference pattern Pr, the first elliptic opening portion 32 and the second elliptic opening portion 34 according to the embodiment of the present invention. The reference pattern Pr can have a plurality of stripes arranged adjacent to each other, which means the predefined granting pattern as mentioned above. The stripes in the detection pattern Pd are deformed due to reflection from the object Ot. The first major axis Ax1 and the first minor axis Ay1 of the first elliptic opening portion 32 can respectively align with the second major axis Ax2 and the second minor axis Ay2 of the second elliptic opening portion 34. Further, a rotation angle of the first elliptic opening portion 32 relative to a stretching direction Ds or an arrangement direction Da of the reference pattern Pr can be preferably the same as or similar to a rotation angle of the second elliptic opening portion 34 relative to a stretching direction Ds′ or an arrangement direction Da′ of the detection pattern Pd. A first included angle between at least one of the first major axis Ax1 and the first minor axis Ay1 of the first elliptic opening portion 32 and any stripe (or the stretching direction Ds) of the reference pattern Pr can be the same as or similar to a second included angle between at least one of the second major axis Ax2 and the second minor axis Ay2 of the second elliptic opening portion 34 and the stretching direction Ds′ of the detection pattern Pd.

The reference pattern Pr and the first elliptic opening portion 32 (or the detection pattern Pd and the second elliptic opening portion 34) are not set on the same plane, so that the first included angle and the second included angle are not marked in FIG. 3. The present invention can define that an angle between one stripe of the reference pattern Pr and at least one of the first major axis Ax1 and the first minor axis Ay1 is the first included angle when the reference pattern Pr is moved toward the first elliptic opening portion 32 along an optical transmission path (such as a left dotted line shown in FIG. 3) in a non-rotatable and nor-deformable manner; an angle between the stretching direction Ds′ of the detection pattern Pd and at least one of the second major axis Ax2 and the second minor axis Ay2 is the second included angle when the detection pattern Pd is moved toward the second elliptic opening portion 34 along the optical transmission path (such as a right dotted line shown in FIG. 3) in a non-rotatable and nor-deformable manner. Besides, an included angle between the arrangement direction Da of the reference pattern Pr and at least one of the first major axis Ax1 and the first minor axis Ay1 can be defined as the first included angle, and an included angle between the arrangement direction Da′ of the detection pattern Pd and at least one of the second major axis Ax2 and the second minor axis Ay2 can be defined as the second included angle.

As shown in FIG. 3, the plurality of stripes of the reference pattern Pr can be stretched in the stretching direction Ds and arranged in the arrangement direction Da. A major axis direction Dx1 of the first major axis Ax1 of the first elliptic opening portion 32 can be crossed by the arrangement direction Da of the reference pattern Pr. A major axis direction Dx2 of the second major axis Ax2 of the second elliptic opening portion 34 can be crossed by the arrangement direction Da of the reference pattern Pr. The crossed angle of the major axis direction Dx1 relative to the arrangement direction Da can preferably be ninety degrees, and the crossed angle of the major axis direction Dx2 relative to the arrangement direction Da can preferably be ninety degrees, or the major axis direction Dx1 and the major axis direction Dx2 can be substantially perpendicular to the arrangement direction Da; however, the prevent invention may have angle error tolerance. A percentage of the angle error tolerance in the present invention can depend on opto-mechanical design of the three dimensional scanning apparatus 10, such as 3-5%, and an actual value of the percentage is not limited to the foresaid embodiment.

The first elliptic opening portion 32 and the second elliptic opening portion 34 can be designed as the fixed aperture opening portion, such as the oval hole formed on the aperture unit. The first elliptic opening portion 32 and the second elliptic opening portion 34 can be further designed as the variable aperture opening portion. The variable aperture opening portion can include a plurality of blades, and position variation in the blades can be used to enlarge or reduce a size of the opening portion, so as to adjust an amount of received light by the aperture unit.

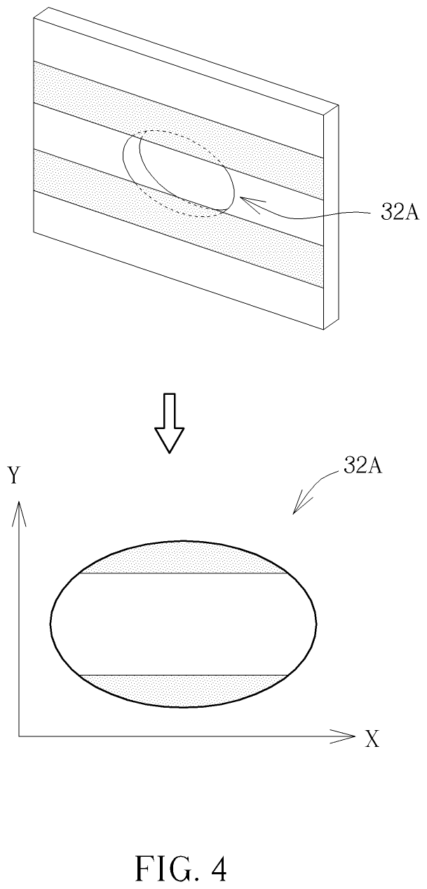

Please refer to FIG. 4. FIG. 4 is a diagram of the first elliptic opening portion 32A according to another embodiment of the present invention. In the foresaid embodiment, the first elliptic opening portion 32 and the second elliptic opening portion 34 are the oval holes formed on the aperture unit. In other possible embodiment, as shown in FIG. 4, an upper part and a lower part of the oval hole can be sheltered to provide a specific opening structure used as the first elliptic opening portion 32A, which still can keep a scanning depth of field of the three dimensional scanning apparatus 10 and increase an intensity of the detection pattern Pd; the second elliptic opening portion can be also designed as the same type. Types of the first elliptic opening portion and the second elliptic opening portion are not limited to the above-mentioned embodiments, and a detailed description is omitted herein for simplicity.

Therefore, the three dimensional scanning apparatus 10 of the present invention can utilize the illumination light source 12 to emit the illumination beam B, and the illumination beam B can be reflected toward the reference pattern generator 18 through the reflector 30, and a programmable function of the reference pattern generator 18 can applied to generate the reference pattern Pr having the predefined pattern. The reference pattern Pr can be reflected toward the object Ot through the first elliptic opening portion 32 and the reflector 22. The object Ot has the undulated contour to result in the detection pattern Pd by reflection, and the detection pattern Pd can be received by the optical receiver 20 through the second elliptic opening portion 34. The optical receiver 20 can compute and analyze difference between the reference pattern Pr and the detection pattern Pd to determine the contour of the object Ot. The optical receiver 20 can have a built-in processor for related computation and analysis; or, the optical receiver 20 may transmit data to an external processor for the computation and the analysis, which depends on the design demand of the three dimensional scanning apparatus 10.

In the embodiment of the present invention, the three dimensional scanning apparatus 10 can set the first elliptic opening portion 32 and the second elliptic opening portion 34 in different types according to the actual demand. For example, for keeping the scanning depth of field of the three dimensional scanning apparatus 10 and increasing the intensity of the detection pattern Pd, the three dimensional scanning apparatus 10 can lengthen at least one of the first major axis Ax1 of the first elliptic opening portion 32 and the second major axis Ax2 of the second elliptic opening portion 34 along the stretching direction Ds of the reference pattern Pr, so as to increase the amount of received light, and optionally not adjust lengths of the first minor axis Ay1 of the first elliptic opening portion 32 and the second minor axis Ay2 of the second elliptic opening portion 34 to keep the scanning depth of field.

For keeping the intensity of the detection pattern Pd and increasing the scanning depth of field of the three dimensional scanning apparatus 10, the three dimensional scanning apparatus 10 can shorten at least one of the first minor axis Ay1 of the first elliptic opening portion 32 and the second minor axis Ay2 of the second elliptic opening portion 34 along the stretching direction Ds of the reference pattern Pr, so as to increase the scanning depth of field, and optionally not adjust the first major axis Ax1 of the first elliptic opening portion 32 and the second major axis Ax2 of the second elliptic opening portion 34 for keeping the intensity of the detection pattern Pd. It should be mentioned that if the first elliptic opening portion 32 and the second elliptic opening portion 34 are the fixed aperture opening portions, the aperture unit whereon the oval hole is formed can be replaced for the meaning of lengthening or shortening the major axis or the minor axis of the elliptic opening portion along the stretching direction Ds of the reference pattern Pr; if the first elliptic opening portion 32 and the second elliptic opening portion 34 are the variable aperture opening portions, the blades are adjusted to lengthen or shorten the major axis or the minor axis of the elliptic opening portion.

Moreover, the ratio of the first major axis Ax1 to the first minor axis Ay1 of the first elliptic opening portion 32, and the ratio of the second major axis Ax2 to the second minor axis Ay2 of the second elliptic opening portion 34 can be preferably ranged between 1.0 and 2.0, which means the major axis is longer than the minor axis and shorter than twice the minor axis. When the minor axis is fixed and the ratio of the major axis to the minor axis is close to 1.0, the intensity of the reference pattern Pr and the detection pattern Pd are slightly increased by lengthening the major axis of the elliptic opening portion; if the ratio of the major axis to the minor axis is close to 2.0, the major axis of the elliptic opening portion can be lengthened to obviously increase the intensity of the reference pattern Pr and the detection pattern Pd. When the major axis is fixed and the ratio of the major axis to the minor axis is close to 1.0, resolution of the reference pattern Pr and the detection pattern Pd are slightly adjusted by shortening the minor axis of the elliptic opening portion; if the ratio of the major axis to the minor axis is close to 2.0, the minor axis of the elliptic opening portion can be shortened to obviously increase the resolution of the reference pattern Pr and the detection pattern Pd.

The three dimensional scanning apparatus of the present invention can be used in the optical molding apparatus in the dental department. A light source cannot put into the mouth of the patient, and an illumination efficiency of an external light source for projecting into the patient's mouth is insufficient, so that the optical system of the optical molding apparatus has to be advanced for preferred detection accuracy. Thus, the three dimensional scanning apparatus of the present invention can adjust the shape, the ratio and/or the size of the opening portion on the aperture unit to keep the scanning depth of field and the intensity of the pattern, so as to acquire the accurate and clear contour of the object.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A three dimensional scanning apparatus of detecting a contour of an object, the three dimensional scanning apparatus comprising:
an illumination light source adapted to emit an illumination beam;
a first elliptic opening portion;
a reference pattern generator adapted to provide a reference pattern via the illumination beam and transmit the reference pattern toward the object through the first elliptic opening portion;
a second elliptic opening portion; and
an optical receiver adapted to receive a detection pattern reflected from the object via the second elliptic opening portion, so as to analyze a difference between the reference pattern and the detection pattern for acquiring the contour.

2. The three dimensional scanning apparatus of claim 1, wherein a difference between a first ratio of a major axis to a minor axis of the first elliptic opening portion and a second ratio of a major axis to a minor axis of the second elliptic opening portion is smaller than a predefined threshold.

3. The three dimensional scanning apparatus of claim 1, wherein a first included angle of one of a major axis and a minor axis of the first elliptic opening portion relative to the reference pattern is the same as or similar to a second included angle of one of a major axis and a minor axis of the second elliptic opening portion relative to the reference pattern.

4. The three dimensional scanning apparatus of claim 1, wherein the reference pattern comprises a plurality of stripes arranged adjacent to each other, and an axis direction of a major axis of the first elliptic opening portion is crossed by an arrangement direction of the plurality of stripes.

5. The three dimensional scanning apparatus of claim 1, wherein the reference pattern comprises a plurality of stripes arranged adjacent to each other, and an axis direction of a major axis of the second elliptic opening portion is crossed by an arrangement direction of the plurality of stripes.

6. The three dimensional scanning apparatus of claim 4, wherein an included angle between the major axis of the first elliptic opening portion and the arrangement direction is equal to ninety degrees, or the major axis of the first elliptic opening portion is perpendicular to the arrangement direction and has an angle error tolerance.

7. The three dimensional scanning apparatus of claim 5, wherein an included angle between the major axis of the second elliptic opening portion and the arrangement direction is equal to ninety degrees, or the major axis of the second elliptic opening portion is perpendicular to the arrangement direction and has an angle error tolerance.

8. The three dimensional scanning apparatus of claim 1, wherein the first elliptic opening portion and the second elliptic opening portion are fixed aperture opening portions or variable aperture opening portions.

9. The three dimensional scanning apparatus of claim 1, wherein the reference pattern comprises a plurality of stripes arranged adjacent to each other, at least one of a major axis of the first elliptic opening portion and a major axis of the second elliptic opening portion is lengthened along a stripe direction of the plurality of stripes, so as to keep a scanning depth of field of the three dimensional scanning apparatus and increase an intensity of the detection pattern.

10. The three dimensional scanning apparatus of claim 1, wherein the reference pattern comprises a plurality of stripes arranged adjacent to each other, at least one of a minor axis of the first elliptic opening portion and a minor axis of the second elliptic opening portion is shortened along an arrangement direction of the plurality of stripes, so as to keep an intensity of the detection pattern and increase a scanning depth of field of the three dimensional scanning apparatus.

11. The three dimensional scanning apparatus of claim 1, wherein the reference pattern generator is an assembly of digital mirror devices, and the reference pattern is a predefined granting pattern generated by the assembly of digital mirror devices.
